(19) United States
(12) Patent Application Publication
(10) Pub. No.: US 2008/0242861 A1
(43) Pub. Date: Oct. 2, 2008

(54) Synthesis of amino-protected cyclohexane-1,4-diyldimethanamine and its derivatives

(76) Inventors: Chi-Feng Yen, Sanjhih Township (TW); Chang-Pin Huang, Sanchong City (TW); Cheng-Kung Hu, Hsinchu City (TW); Ming-Chen Chou, Banchaw City (TW); Chi-Hsin Richard King, Centerville, OH (US)

Correspondence Address: OCCHIUTI ROHLICEK & TSAO, LLP, 10 FAWCETT STREET, CAMBRIDGE, MA, 02138, US

(21) Appl. No.: 11/732,094

(22) Filed: Apr. 2, 2007

(51) Int. Cl. C07D 239/72; C07C 209/34; C07C 209/68; C07C 211/34; C07D 207/06; C07D 217/02; C07D 239/24; C07D 239/30

(52) U.S. Cl. 544/291; 544/292; 544/323; 544/326; 546/139; 546/146; 548/537; 564/445; 564/448; 564/462; 564/87

(57) ABSTRACT

This invention relates to methods of preparing the compounds of formula (V):

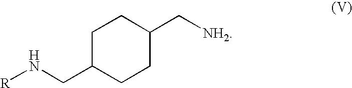

Each variable in this formula is defined in the specification.

### BACKGROUND

Amino-protected cyclohexane-1,4-diyldimethanamine compounds have been used as intermediates for synthesizing CXCR4 antagonists, thrombin inhibitors, MCH receptor and Y5 receptor antagonists, and GPR antagonists. See, e.g., WO 95/23609, WO 03/028641, WO 97/46250, and EP 1295867. However, conventional methods of manufacturing these intermediates involve toxic chemicals and tedious purification processes. There is a need for a safer and simpler method for synthesizing this intermediate in a high yield.

### SUMMARY

This invention relates to processes of preparing amino-protected cyclohexane-1,4-diyldimethanamine compounds. These compounds can be used to prepare drugs that are effective in treating a variety of diseases, including inflammatory/immune diseases, developmental/degenerative diseases, and tissue injuries.

Thus, in one aspect, this invention features a chemical synthetic method of reacting a compound of formula (III),

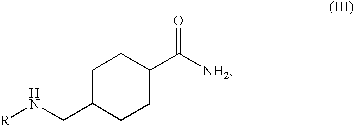

with a dehydrating agent (e.g., trifluoroacetic anhydride) to give a compound of formula (IV),

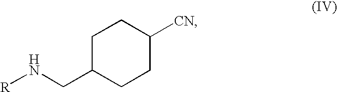

in which R is an amino-protecting group. Examples of amino-protecting group include tert-butoxycarbonyl, benzyloxycarbonyl, acetyl, and phenylcarbonyl.

A compound of formula (III) can be obtained by reacting a compound of formula (II),

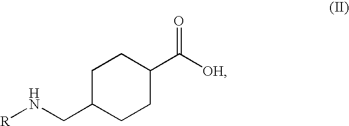

with a chloroformate (e.g., ethyl carbonochloridate) to give an anhydride, followed by reacting the anhydride with an amidation agent (e.g., ammonia). When the amino-protecting group R is tert-butoxycarbonyl, a compound of formula (II) can be obtained by reacting a compound of formula (I),

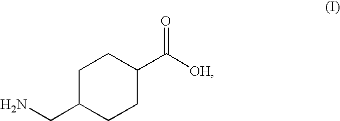

with di-tert-butyl carbonate.

The chemical synthetic method can further include reacting a compound of formula (IV) with a reducing agent (e.g., hydrogen gas) to give a compound of formula (V),

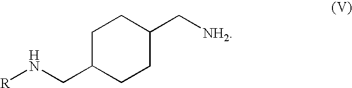

In another aspect, this invention features a chemical synthetic method that includes reacting a compound of formula (IV), which can be obtained by the method described above, with a reducing agent (e.g., hydrogen gas) to give a compound of formula (V).

The details of one or more embodiments of the invention are set forth in the description below. Other features, objects, and advantages of the invention will be apparent from the description and from the claims.

### DETAILED DESCRIPTION

This invention relates to methods of preparing compounds of formula (V). Specifically, these compounds can be prepared by the approach shown in Scheme 1 below.

As shown in Scheme 1, a compound of formula (V) can be prepared from a compound of formula (I) via four steps:

Step (1): reacting the compound of formula (I) with an amino-protecting agent (e.g., di-tert-butyldicarbonate) to give a compound of formula (II);

Step (2): reacting the compound of formula (II) with a chloroformate (e.g., ethyl chloroformate) to give an anhydride, followed by reacting the anhydride with an amidation agent, e.g., ammonia (g)/ammonium hydroxide, to give a compound of formula (III);

Step (3): reacting the compound of formula (III) with a dehydrating agent (e.g., trifluoroacetic anhydride) to give a compound of formula (IV); and

Step (4): reacting the compound of formula (IV) with a reducing agent (e.g., hydrogen gas) to give a compound of formula (V).

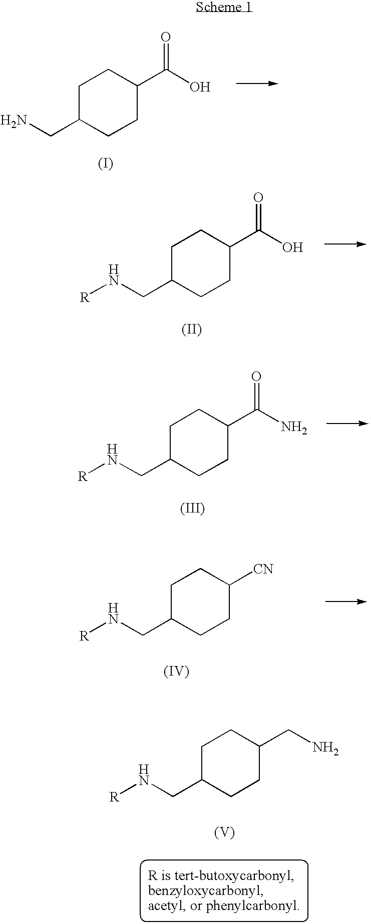

Compounds of formula (I) can be purchased from a commercial source, such as Sigma-Aldrich (St. Louis, Mo.), or prepared by methods known in the art. Referring to step (1), the compound of formula (I) can react with any suitable amino-protecting agents to form a compound of formula (II). Examples of suitable amino-protecting agents include di-tert-butyldicarbonate (i.e., (Boc)₂O), imidazole-Boc, benzyloxycarbonyl chloride, acetyl chloride, or phenylcarbonyl chloride. Exemplary amino-protecting groups include t-butoxycarbonyl (t-Boc), benzyloxycarbonyl, acetyl, or phenylcarbonyl. t-Boc is preferred among the four exemplary amino-protecting groups above since acetyl is difficult to remove during the preparation of a final product (e.g., a drug), and benzyloxycarbonyl and phenylcarbonyl are readily removed during the subsequent reduction reaction in step (4), thereby losing their protection function. Step (1) is typically carried out in the presence of a base (e.g., a NaHCO₃ aqueous solution).

Referring to step (2), the two reactions (i.e., the reaction between the compound of formula (II) and chlorofomate, followed by treatment of the amidation agent) can be carried out in a one-pot reaction, thereby eliminating the need to isolate and purify the intermediate anhydride and increasing the yield of the compound of formula (III). Preferably, these two reactions are carried out at a low temperature (e.g., no higher than −10° C.) to reduce generation of by-products and to obtain the compound of formula (III) in a high yield.

Referring to step (3), trifluoroacetic anhydride is preferred over other dehydrating agents, such as trifluoroacetic acid, phosphorus oxychloride, methanesulfonyl chloride, trifluoromethanesulfonyl chloride, or trifluoromethanesulfonic anhydride. Reactions with these other dehydrating agents either give the compound of formula (IV) with low yields or require tedious purification processes.

Referring to step (4), hydrogen gas is preferred over other reducing agents, such as LiAlH₄, which gives the compound of formula (V) with a low yield. The reduction reaction in step (4) is typically carried out in the presence of a catalyst. Unexpectedly, use of a combination of the Raney-nickel catalyst and Pd/C results in production of the compound of formula (V) with a very high yield (e.g., at least 90% or at least 95%), while use of either the Raney-nickel catalyst or Pd/C alone only gives the compound of formula (V) with a low yield.

The synthetic route described in Scheme 1 avoids use of highly explosive azide compounds (e.g., sodium azide) typically used in convention methods and therefore is much safer and cleaner than those methods.

A compound of formula (V) can be used as an intermediate to prepare a variety of compounds with therapeutic efficacy via methods known in the art. For example, it can be used to prepare certain pyrimidine compounds for treating an inflammatory or immune disease, a developmental or degenerative disease, or a tissue injury. Examples of such pyrimidine compounds have been described in commonly owned co-pending U.S. Application Serial No. 20060293324.

As another example, Scheme 2 below shows a synthetic route of preparing the compound of formula (VIII), which can be used for treating obesity or obesity related disorders, anxiety, or depression. Specifically, as shown in Scheme 2, a compound of formula (V) can react with (2-chloro-quinazolin-4-yl)-dimethyl-amine via a substitution reaction to give a compound of formula (VI). The amino-protecting group R of the compound of formula (VI) can then be removed (e.g., under an acidic condition) to give a compound of formula (VII), which can react with 4-bromo-2-trifluoromethoxy-benzensulfonyl chloride to give a compound of formula (VIII).

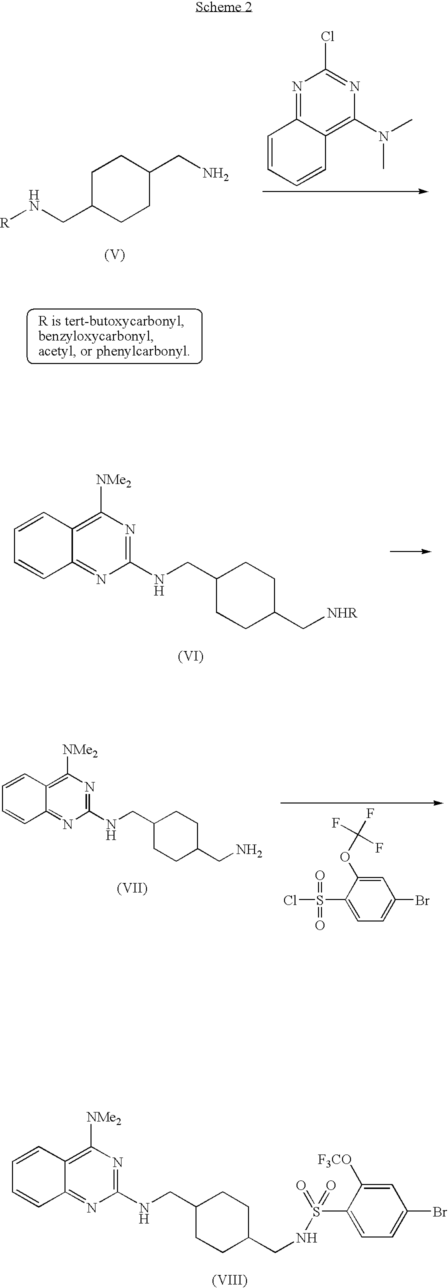

As another example, Scheme 3 below shows a synthetic route of preparing the compound of formula (IX), which can be used as an anti-thrombotic agent. Specifically, as shown in Scheme 3, the compound of formula (IX) can be prepared by reacting a compound of formula (V) with 1-(2-(tert-butoxycarbonylamino)-3-phenylpropanoyl)-pyrrolidine-2-carboxylic acid via an amidation reaction, followed by removal of the amino-protecting groups R and t-Boc (e.g., under an acidic condition).

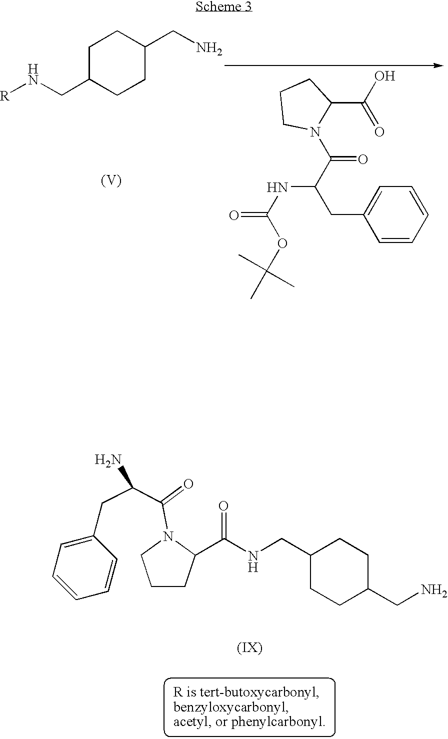

As another example, Scheme 4 below shows a synthetic route of preparing the compound of formula (XIII), which can be used as an agonist or antagonist of Y5 receptor for treating obesity, bulimia, or anorexia. Specifically, as shown in Scheme 4, the compound of formula (V) can react with 2-nitrobenzene-1-sulfonyl chloride via a substitution reaction to give a compound of formula (X). The amino-protecting group R of the compound of formula (X) can then be removed (e.g., under an acidic condition) to give a compound of formula (XI). The compound of formula (XI) can subsequently react with isoquinoline-3-carboxylic acid via an amidation reaction to give a compound of formula (XII), which can then react with a reducing agent (e.g., borane) to give a compound of formula (XIII).

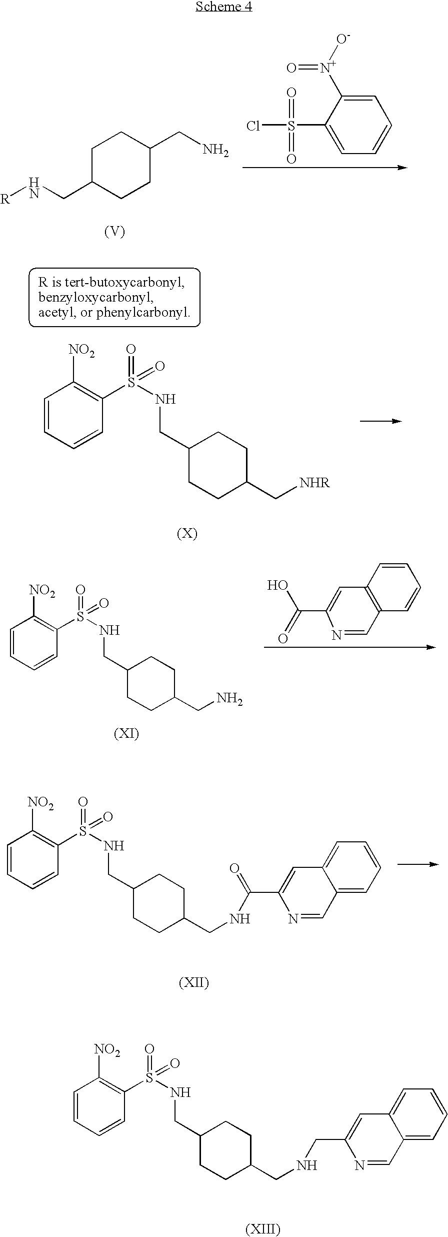

As another example, Scheme 5 below shows a synthetic route of preparing the compound of formula (XVI), which can be used as a PKC-theta inhibitor for treating PKC-theta mediated disorders, such as immunological disorders and type II diabetes. Specifically, as shown in Scheme 5, the compound of formula (V) can react with 2,4-dichloro-5-nitropyrimidine via a substitution reaction to give a compound of formula (XIV), which can react with (2,5-dichlorophenyl)methanamine via another substitution reaction to give a compound of formula (XV). The amino-protecting group R of the compound of formula (XV) can subsequently be removed (e.g., under an acidic condition) to give a compound of formula (XVI).

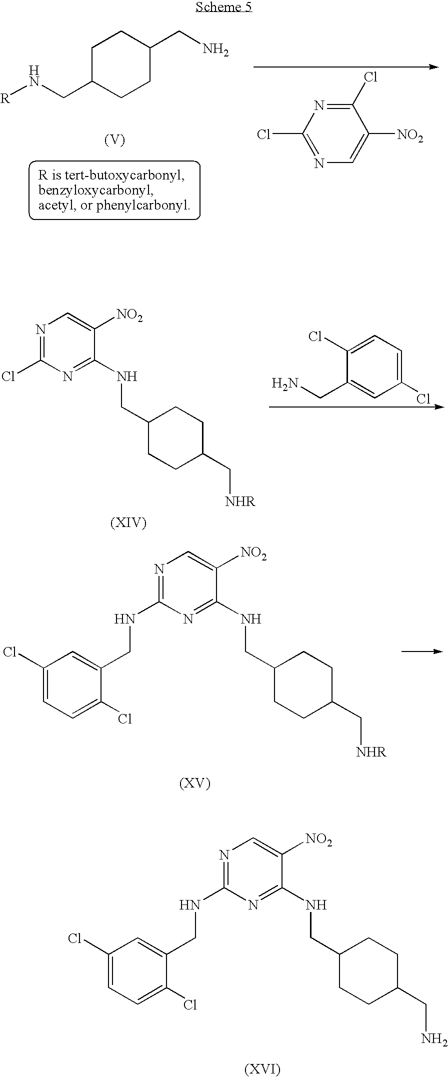

Compound synthesized by the methods described above can be further purified by column chromatography, high-pressure liquid chromatography, recrystallization, or any other suitable techniques.

Other compounds can be prepared using other suitable starting materials through the synthetic routes set forth above. The methods described above may also include additional steps, either before or after the steps described above, to add or remove suitable protecting groups in order to ultimately allow synthesis of the polyamine compounds. In addition, various synthetic steps may be performed in an alternate sequence or order to give the desired compounds.

The compounds used in or obtained from the methods described above may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

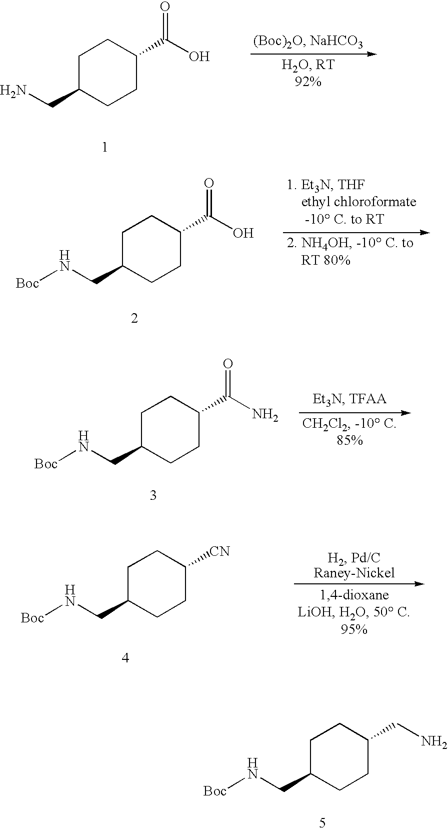

Water (10.0 L) and (Boc)₂O (3.33 Kg, 15.3 mol) were added to a solution of trans-4-aminomethyl-cyclohexanecarboxylic acid (Compound 1, 2.0 Kg, 12.7 mol) and sodium bicarbonate (2.67 Kg, 31.8 mol). The reaction mixture was stirred at ambient temperature for 18 hours. The aqueous layer was acidified with concentrated hydrochloric acid (2.95 L, PH=2) and then filtered. The resultant solid was collected, washed three times with water (15 L), and dried in a hot box (60° C.) to give trans-4-(tert-butoxycarbonylamino-methyl)-cyclo-hexanecarboxylic acid (Compound 2, 3.17 Kg, 97%) as a white solid. R_{f}=0.58 (EtOAc). LC-MS m/e 280 (M+Na⁺). ¹H NMR (300 MHz, CDCl₃) δ 4.58 (brs, 1H), 2.98 (t, J=6.3 Hz, 2H), 2.25 (td, J=12, 3.3 Hz, 1H), 2.04 (d, J=11.1 Hz, 2H), 1.83 (d, J=11.1 Hz, 2H), 1.44 (s, 9H), 1.35˜1.50 (m, 3H), 0.89˜1.03 (m, 2H). ¹³C NMR (75 MHz, CDCl₃) δ 181.31, 156.08, 79.12, 46.41, 42.99, 37.57, 29.47, 28.29, 27.96. M.p. 134.8˜135.0° C.

A suspension of Compound 2 (1.0 Kg, 3.89.mol) in THF (5 L) was cooled at −10° C. and triethyl amine (1.076 L, 7.78 mol) and ethyl chloroformate (0.441 L, 4.47 mol) were added below −10° C. The reaction mixture was stirred at ambient temperature for 3 hours. The reaction mixture was then cooled at −10° C. again and NH₄OH (3.6 L, 23.34 mol) was added below −10° C. The reaction mixture was stirred at ambient temperature for 18 hours and filtered. The solid was collected and washed three times with water (10 L) and dried in a hot box (60° C.) to give trans-4-(tert-butoxycarbonyl-amino-methyl)-cyclohexanecarboxylic acid amide (Compound 3, 0.8 Kg, 80%) as a white solid. Rf=0.23 (EtOAc). LC-MS m/e 279, M+Na⁺. ¹H NMR (300 MHz, CD₃D) δ 6.63 (brs, 1H), 2.89 (t, J=6.3 Hz, 2H), 2.16 (td, J=12.2, 3.3 Hz, 1H), 1.80˜1.89 (m, 4H), 1.43 (s, 9H), 1.37˜1.51 (m, 3H), 0.90˜1.05 (m, 2H). ¹³C NMR (75 MHz, CD₃OD) δ 182.26, 158.85, 79.97, 47.65, 46.02, 39.28, 31.11, 30.41, 28.93. M.p. 221.6˜222.0° C.

A suspension of Compound 3 (3, 1.2 Kg, 4.68 mol) in CH₂Cl₂ (8 L) was cooled at −10° C. and triethyl amine (1.3 L, 9.36 mol) and trifluroracetic anhydride (0.717 L, 5.16 mol) were added below −10° C. The reaction mixture was stirred at −10° C. for 3 hours. After water (2.0 L) was added, the organic layer was separated and washed with water (3.0 L) twice. The organic layer was then passed through silica gel and concentrated. The oil isolated was crystallized by methylene chloride. The crystals were washed with hexane to give trans-(4-cyano-cyclohexylmethyl)-carbamic acid tert-butyl ester (Compound 4, 0.95 Kg, 85%) as a white crystal. R_{f}=0.78 (EtOAc). LC-MS m/e 261, M+Na⁺. ¹H NMR (300 MHz, CDCl₃) δ 4.58 (brs, 1H), 2.96 (t, J=6.3 Hz, 2H), 2.36 (td, J=12, 3.3 Hz, 1H), 2.12 (dd, J=13.3, 3.3 Hz, 2H), 1.83 (dd, J=13.8, 2.7 Hz, 2H), 1.42 (s, 9H), 1.47˜1.63 (m, 3H), 0.88˜1.02 (m, 2H). ¹³C NMR (75 MHz, CDCl₃) δ 155.96, 122.41, 79.09, 45.89, 36.92, 29.06, 28.80, 28.25, 28.00. M.p. 100.4˜100.6° C.

Compound 4 (1.0 Kg, 4.196 mol) was dissolved in a mixture of 1,4-dioxane (8.0 L) and water (2.0 L). To the reaction mixture were added lithium hydroxide monohydrate (0.314 Kg, 4.191), Raney-nickel (0.4 Kg, 2.334 mol), and 10% palladium on carbon (0.46 Kg, 0.216 mol) as a 50% suspension in water. The reaction mixture was stirred under hydrogen atmosphere at 50° C. for 20 hours. After the catalysts were removed by filtration and the solvents were removed in vacuum, a mixture of water (1.0 L) and CH₂Cl₂ (0.3 L) was added. After phase separation, the organic phase was washed with water (1.0 L) and concentrated to give trans-(4-aminomethyl-cyclohexylmethyl)-carbamic acid tert-butyl ester (Compound 5, 0.97 Kg, 95%) as pale yellow thick oil. R_{f}=0.20 (MeOH/EtOAc=9/1). LC-MS m/e 243, M+H⁺. ¹H NMR (300 MHz, CDCl₃) δ 4.67 (brs, 1H), 2.93 (t, J=6.3 Hz, 2H), 2.48 (d, J=6.3 Hz, 2H), 1.73˜1.78 (m, 4H), 1.40 (s, 9H), 1.35 (brs, 3H), 1.19˜1.21 (m, 1H), 0.77˜0.97 (m, 4H). ¹³C NMR (75 MHz, CDCl₃) δ 155.85, 78.33, 48.27, 46.38, 40.80, 38.19, 29.87, 29.76, 28.07.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1-2. (canceled)

3. A chemical synthetic method, comprising
reacting a compound of formula (III) 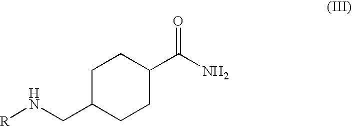
with a dehydrating agent to give a compound of formula (IV) 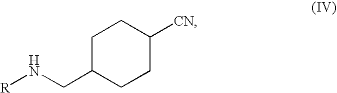
in which R is an amino-protecting group, and
reacting the compound of formula (IV) with a reducing agent to give a compound of formula (V)

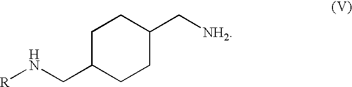

4. The method of claim 3, wherein the reducing agent is hydrogen gas.

5-9. (canceled)

10. The method of claim 3, further comprising reacting the compound of formula (V) with 2-chloro-quinazolin-4-yl)-dimethyl-amine to give a compound of formula (VI)

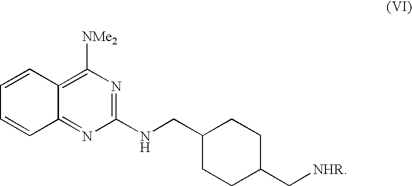

11. The method of claim 10, further comprising removing the amino-protecting group R of the compound of formula (VI) to give a compound of formula (VII)

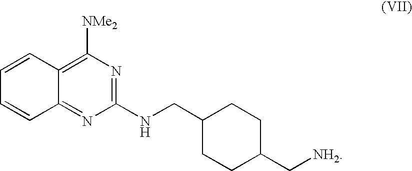

12. The method of claim 11, further comprising reacting the compound of formula (VII) with 4-bromo-2-trifluoromethoxy-benzensulfonyl chloride to give a compound of formula (VIII)

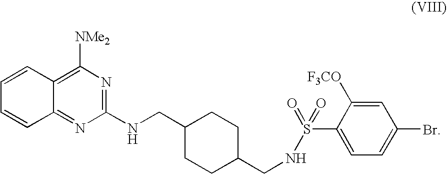

13. The method of claim 3, further comprising reacting the compound of formula (V) with 1-(2-(tert-butoxycarbonylamino)-3-phenylpropanoyl)pyrrolidine- 2 -carboxylic acid to give an amide, followed by removing the amino-protecting group R and the t-Boc group to give a compound of formula (IX)

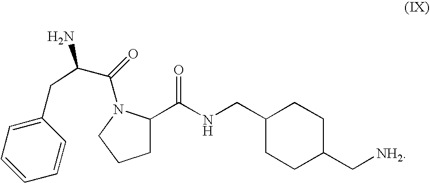

14. The method of claim 3, further comprising reacting the compound of formula (V) with 2-nitrobenzene-1-sulfonyl chloride to give a compound of formula (X)

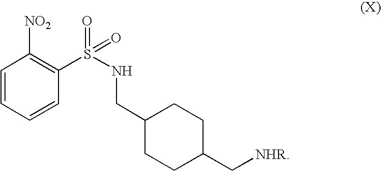

15. The method of claim 14, further comprising removing the amino-protecting group R of the compound of formula (X) to give a compound of formula (XI)

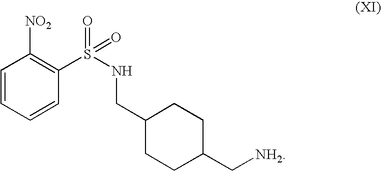

16. The method of claim 15, further comprising reacting the compound of formula (XI) with isoquinoline-3-carboxylic acid to give a compound of formula (XII)

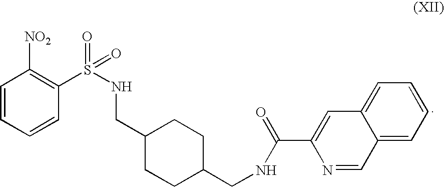

17. The method of claim 16, further comprising reacting the compound of formula (XII) with a reducing agent to give a compound of formula (XIII)

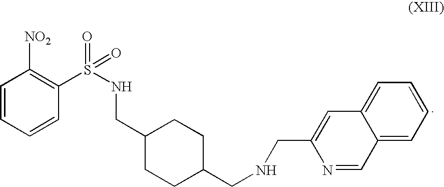

18. The method of claim 3, further comprising reacting the compound of formula (V) with 2,4-dichloro-5-nitropyrimidine to give a compound of formula (XIV)

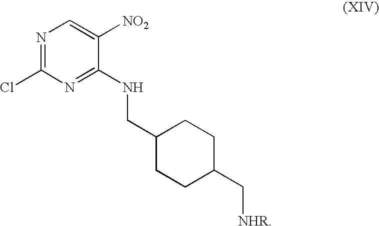

19. The method of claim 18, further comprising reacting the compound of formula (XIV) with ( 2,5 -dichlorophenyl)methanamine to give a compound of formula (XV)

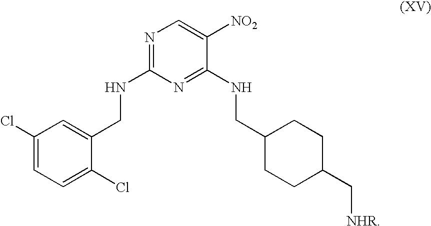

20. The method of claim 19, further comprising removing the amino-protecting group R of the compound of formula (XV) to give a compound of formula (XVI)

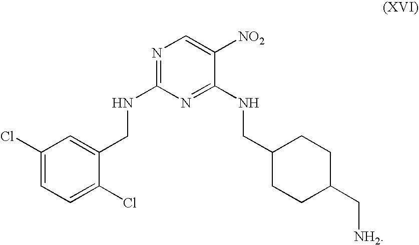

21. A chemical synthetic method, comprising reacting a compound of formula (IV) 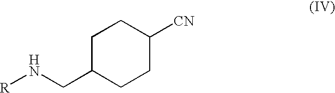
with a reducing agent to give a compound of formula (V) 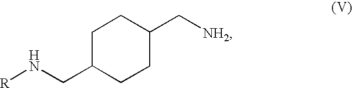
wherein R is an amino-protecting group.

22. The method of claim 21, wherein the reducing agent is hydrogen gas.

23. The method of claim 21, wherein the compound of formula (IV) is obtained by reacting a compound of formula (III) 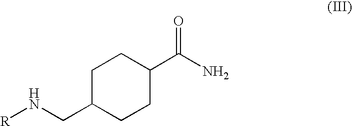
with a dehydrating agent.

24. The method of claim 23, wherein the dehydrating agent is trifluoroacetic anhydride.

25. The method of claim 23, wherein the compound of formula (III) is obtained by reacting a compound of formula (II) 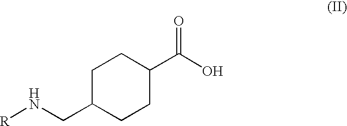
with a chloroformate to give an anhydride, followed by reacting the anhydride with an amidation agent.

26. The method of claim 25, wherein the amidation agent is ammonia (g)/ammonium hydroxide.

27. The method of claim 21, wherein the amino-protecting group R is tert-butoxycarbonyl, benzyloxycarbonyl, acetyl, or phenylcarbonyl.

28. The method of claim 27, wherein the amino-protecting group R is tert-butoxycarbonyl.

29. The method of claim 28, wherein the compound of formula (II) is obtained by reacting a compound of formula (I) 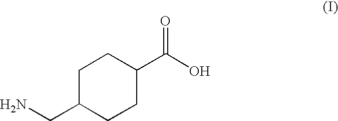
with di-tert-butyl carbonate.

30. The method of claim 21, further comprising reacting the compound of formula (V) with 2-chloro-quinazolin-4-yl)-dimethyl-amine to give a compound of formula (VI)

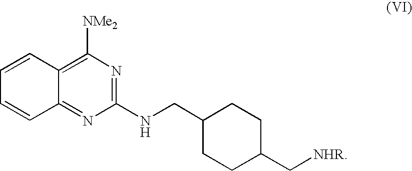

31. The method of claim 30, further comprising removing the amino-protecting group R of the compound of formula (VI) to give a compound of formula (VII)

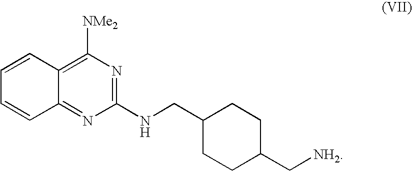

32. The method of claim 31, further comprising reacting the compound of formula (VII) with 4-bromo-2-trifluoromethoxy-benzensulfonyl chloride to give a compound of formula (VIII)

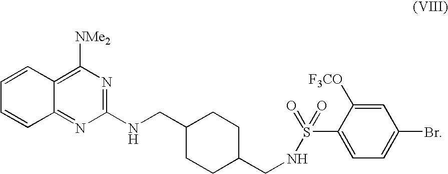

33. The method of claim 21, further comprising reacting the compound of formula (V) with 1-(2-(tert-butoxycarbonylamino)-3-phenylpropanoyl)pyrrolidine-2-carboxylic acid to give an amide, followed by removing the amino-protecting group R and the t-Boc group to give a compound of formula (IX)

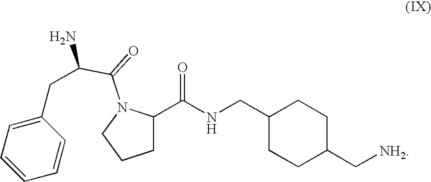

34. The method of claim 21, further comprising reacting the compound of formula (V) with 2-nitrobenzene-1-sulfonyl chloride to give a compound of formula (X)

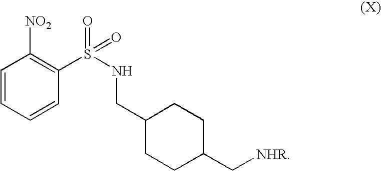

35. The method of claim 34, further comprising removing the amino-protecting group R of the compound of formula (X) to give a compound of formula (XI)

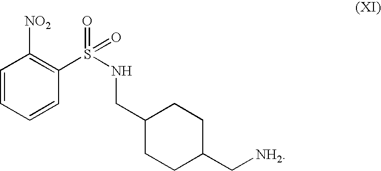

36. The method of claim 35, further comprising reacting the compound of formula (XI) with isoquinoline-3-carboxylic acid to give a compound of formula (XII)

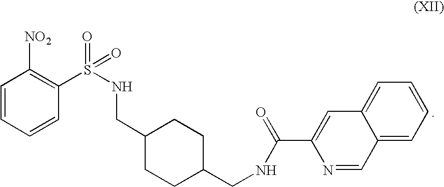

37. The method of claim 36, further comprising reacting the compound of formula (XII) with a reducing agent to give a compound of formula (XIII)

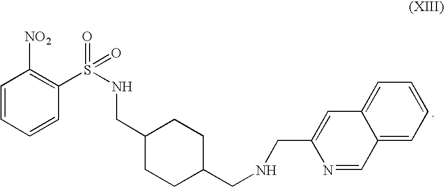

38. The method of claim 21, further comprising reacting the compound of formula (V) with 2,4-dichloro-5-nitropyrimidine to give a compound of formula (XIV)

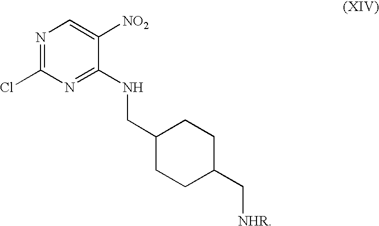

39. The method of claim 38, further comprising reacting the compound of formula (XIV) with (2,5-dichlorophenyl)methanamine to give a compound of formula (XV)

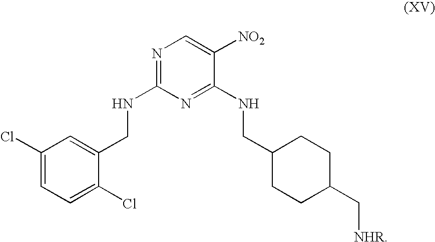

40. The method of claim 39, further comprising removing the amino-protecting group R of the compound of formula (XV) to give a compound of formula (XVI) [chemical structure]
